(19) United States
(12) Patent Application Publication
(10) Pub. No.: US 2011/0184272 A1
(43) Pub. Date: Jul. 28, 2011

(54) Portable Diagnostic Device for Precancerous Lesion of Cervical Cancer

(76) Inventors: Kun Zeng, Shanghai (CN); Zhen Fen Yu, Shanghai (CN)

(21) Appl. No.: 13/054,415

(22) PCT Filed: Jan. 12, 2009
(86) PCT No.: PCT/CN2009/070098

§ 371 Date: Apr. 11, 2011

(30) Foreign Application Priority Data
Jul. 15, 2008 (CN) 200810131630.7

(51) Int. Cl. A61B 5/05; A61B 17/42

(52) U.S. Cl. 600/407; 606/119

(57) ABSTRACT

A portable diagnostic device for cervical precancerous lesions is disclosed. The device includes an inherent fluorescence detection system for cervical cancer, a cervix acetic acid coloring detection and an image collection system. The inherent fluorescence detection system for cervical cancer has a light source and a switch, which consists of a excitation light source that generates fluorescence and a cold light source that generates white light, both the excitation light source and the cold light source are installed in the bowl-shaped reflector with specific focal length. The cervix acetic acid coloring detection system has an acetic acid pool, a spraying tube that connects with the acetic acid pool, a spraying tube moving device, and a spraying switch. The image collection system has a weak light CCD, which is installed in the center of the bowl-shaped reflector. The image collection system is connected to the external image processing system which process the images collected by the image collection system. Use the white light and excitation light to irradiate the cervix in turn and collect the reflected images from the cervix. Spray acetic acid on cervix and use the white light and excitation light to irradiate the cervix in turn after waiting for a period of time. Collect the reflected images from the cervix which has been sprayed the acetic acid.

### FIELD OF THE INVENTION

The present invention involves diagnostic device for precancerous lesions, particularly, involves diagnostic device for fast screening or standard detection of precancerous lesions of cervical cancer.

### BACKGROUND

One of the main reasons of the high mortality of cancer over the world is that there is no great breakthrough in clinical diagnosis, of which the method is still morphological, for instance the doctor's experience, colposcope and other endoscopes, ultrasound, CT, MRI, etc. This kind of diagnostic methods is difficult to differentially diagnose a tiny cancer smaller than 0.5 cm. This is the case of epithelial cancers which rise from the epithelia (mucous membrane). They still haven't broken through the epithelia (mucous membrane) and haven't formed all characters of a cancer, so they are called the precancerous lesions. Taking precancerous lesions of the cervical cancer as an example (abbreviate as CIN, cervical intraepithelial neoclassic). CIN I or CIN II takes place in the lower ⅓ level of the mucous membrane as shown in FIG. 1, which does not have any sign of the disease on the surface of epithelia (mucous membrane), so the diagnostic methods using the gross morphology are unable to detect the exact site of precancerous lesions. The detection of CIN is the basis to give appropriate therapy and to ensure the success of the treatment. It is also the only way to block the development of cervical cancer effectively.

There are quite a few methods and devices used to diagnose the cervical cancer over the world at present. Widely used Colposcope is actually a kind of magnifying glass for observing the changes of shape, color and blood vessel of the epithelia surface of the cervix. However CIN is the cellular and biochemical change inside the epithelia (mucous membrane), which will hardly demonstrate valuable information on epithelia (mucous membrane). So the colposcope is valuable in the detection and diagnosis for the cervical cancer (preinvasive cancer) or the cancer in situ (intraepithelial cancer), but its detection rate for CIN is relatively low.

Observation of VIA after acetic acid coloring with naked eyes has some value for CIN. The well differentiated epithelia and poorly differentiated epithelia will be shown as white patches on the acetic acid smeared cervix. It's possible that the white patch is CIN, but its positive detection rate for CIN is just about 40%.

### SUMMARY

The present invention is intended to provide a kind of portable diagnostic device for cervical precancerous lesions that can detect CIN with high efficiency and low cost. It is a diagnostic device which has integrated both the inherent fluorescence image system and cervix acetic acid coloring system.

A first type of device according to the present invention includes: an inherent fluorescence detection system for cervical cancer, with a light source and a switch, which consists of a excitation light source that generates fluorescence and a cold light source that generates white light, both the excitation light source and the cold light source being installed in a bowl-shaped reflector with specific focal length; a cervix acetic acid coloring detection system with an acetic acid pool, a spraying tube that connects with the acetic acid pool, a spraying tube moving device to move the spraying tube, and a spraying switch to control the spraying tube; the inherent fluorescence detection system for cervical precancerous lesions using the white light and excitation light to irradiate the cervix in turn; the cervix acetic acid coloring system spraying acetic acid on cervix, using the white light and excitation light to irradiate the cervix in turn after waiting for a period of time. This device is suitable for quick screening.

A second type of device according to the present invention includes: an inherent fluorescence detection system for cervical cancer, with a light source and a switch, which consists of a excitation light source that generates fluorescence and a cold light source that generates white light, both the excitation light source and the cold light source being installed in a bowl-shaped reflector with specific focal length; a cervix acetic acid coloring detection system with an acetic acid pool, a spraying tube that connects with the acetic acid pool, a spraying tube moving device to move the spraying tube, and a spraying switch to control the spraying tube; an image collection system with a weak light CCD, which is installed in the center of the bowl-shaped reflector and collects the reflected light from the cervix, the image collection system being connected to the external image processing system which then process the image collected; the inherent fluorescence detection system for cervical precancerous lesions using the white light and excitation light to irradiate the cervix in turn; the image collection system collecting reflected images from the cervix induced by white and excitation light then transmitting these images to the external image processing system; the cervix acetic acid coloring system spraying acetic acid on cervix and using the white light and excitation light to irradiate the cervix in turn after waiting for a period of time; the image collection system collecting reflected images from the cervix induced by acetic acid spray then transmitting these images to the external image processing system. This device is suitable for standard examination.

The device is fixed on a pistol shape stand. The pistol shape stand consists of a horizontal part and a handle. The back end of the horizontal part connects with the upper end of the handle. The bowl-shaped reflector is at the front of the horizontal part. The light sources are installed on the bowl-shaped reflector with same distance apart to form a ring. The acetic acid pool is inside the handle. The spraying tube extends from acetic acid pool to the top of the handle. After passing a direction switch the spraying tube continues along beneath the horizontal part to extend to the front end inside the horizontal part. The spraying tube moving device can be installed on the horizontal part with some flexibility. The spraying tube moving device reaches beyond the horizontal part and connects with the spraying tube inside the horizontal part. The spraying tube moving device can drive the front end of the protruded horizontal part. The light source switches are installed on the handle, which include the switches of excitation light source and cold light source. Spray switch is installed in the handle.

The wavelength of excitation light source is 360-440 nm. The excitation light source includes laser instrument that can produce a spectrum range of 360-440 nm, LED that can produce a spectrum range of 360-440 nm, high-pressure mercury light that can produce a spectrum range of 360-440 nm, or Xenon light that can produce a spectrum range of 360-440 nm. The excitation light source directs the light into the bowl-shaped reflector through quartz optic fiber, liquid optic fiber or ordinary optic fiber.

The bowl-shaped reflector surface is covered by hard or soft membrane that can reflect ultraviolet light.

The external image processing system includes: an interface circuit, being connected to the weak light CCD in the image collection system; a CPU being connected to the interface circuit for receiving the images collected by the weak light CCD; an image analysis device being connected to the CPU for analyzing the images that are transmitted by CPU; an image process device being connected to the image analysis device for processing the analyzed images; a storage device being connected to the image processing device for storing the images and the processed results; an output device for outputting the images and processed results.

A third type of device according to the present invention includes: an inherent fluorescence detection system for cervical cancer, with a light source and a switch, which consists of a excitation light source that generates fluorescence and a cold light source that generates white light, both the excitation light source and the cold light source being installed in a bowl-shaped reflector with specific focal length, the light from the light source being focused on the cervix by the bowl-shaped reflector; cotton sticks or cotton balls to smear the acetic acid to the cervix; an image collection system with a weak light CCD, which is installed in the center of the bowl-shaped reflector to collect the light reflected from the cervix, the image collection system being connected to the external image processing system which then process the image collected; the inherent fluorescence detection system for cervical precancerous lesions using the white light and excitation light to irradiate the cervix in turn; the image collection system collecting reflected images from the cervix induced by white and excitation light then transmitting these images to the external image processing system; use the cotton sticks or cotton balls to smear acetic acid on cervix, and after waiting for a period of time, use the white light and excitation light to irradiate the cervix in turn; the image collection system collecting reflected images from the acetic acid applied cervix induced by white and excitation light and then transmitting these images to the external image processing system. This device can be used for standard examination.

In the above devices, the excitation light source is excitation light LED; the cold light source is white light LED. Excitation light LED and white light LED are installed in separated darkrooms of the portable diagnostic device for cervical precancerous lesions, respectively. The input end of a fork-like quartz or liquid optic fiber is connected to the excitation light LED and the white light LED, respectively. The output end of the fork-like quartz or liquid optic fiber is covered by the metal or the plastic overcoat.

A fourth type of device according to the present invention includes: an inherent fluorescence detection system for cervical cancer, with a light source and a switch, which consists of a excitation light source that generates fluorescence and a cold light source that generates white light, the input end of the excitation light source and the cold light source being connected to the quartz or liquid optic fiber covered by the metal or plastic overcoat, the output end of the quartz and liquid optic fiber irradiating straight to the cervix; a cervix acetic acid coloring detection system with an acetic acid pool, a spraying tube that connects with the acetic acid pool, a spraying tube to move the spray tube, and a spraying switch to control the spraying tube; the inherent fluorescence detection system for cervical precancerous lesions using the white light and excitation light to irradiate the cervix in turn; the cervix acetic acid coloring system spraying acetic acid on cervix and using the white light and excitation light to irradiate the cervix in turn after waiting for a period of time.

The present invention combines the inherent fluorescence image detection and cervix acetic-acid coloring detection. The detection rate of CIN is greatly improved to about 80%. The diagnosis made with this invention is generally based on the objective evidence so there is little reliance on experience. Even the junior doctors can diagnose CIN accurately. Moreover, it is not expensive and can be quick to make a diagnosis. The cost to examine a patient is only 2 Yuan and time of examination is probably 5 minutes. This invention of portable diagnostic device can detect more cervical precancerous lesions, which may be treated with standard therapy so that its development to cervical cancer can be blocked. Consequently the incidence of cervical cancer will be reduced.

### BRIEF DESCRIPTION OF THE DRAWING(S)

The above or other features, natures or advantages of the present invention will be more apparent to the skilled person in the art by the following descriptions of the embodiments accompanying with the drawings, the same sign reference indicates the identical features throughout the description, and wherein:

FIG. 1 illustrates a fluorescence spectrum of biological molecule that can be detected inside the epithelia;

FIG. 2 illustrates the transformation of the cells inside epithelia;

FIG. 3 illustrates the formation and evolution of the cervical cancer;

FIG. 4 illustrates the early indications of the chemical environment around the host during the transformation of the atypical cells;

FIG. 5 illustrates the mechanism of the generation of fluorescence;

FIG. 6 illustrates the structure of the first type of the diagnostic device for cervical precancerous lesions of this invention;

FIG. 7 illustrates the structure of the external image processing systems of this invention;

FIG. 8 illustrates the structure of the second type of the diagnostic device for cervical precancerous lesions of this invention;

FIGS. 9a and 9b illustrate the structure of the third type of the diagnostic device for cervical precancerous lesions of this invention;

FIG. 10a and FIG. 10b illustrate the structure of the fourth type of the diagnostic device for cervical precancerous lesions of this invention.

### DETAILED DESCRIPTION

85% of the tumours are originated from the epithelia. Cervical cancer is one among them. Mucous membrane of the cervix is located between 0.6 mm-1.2 mm of the epithelium. The abnormality in the epithelium is occurred gradually. It's expressed with the biochemical information but not expressed with the form of morphology. Therefore the information of precancerous lesions can't be obtained by the physical methods like Ultrasound, X-ray, CT and MRI, let alone the observation of CIN information with naked eyes.

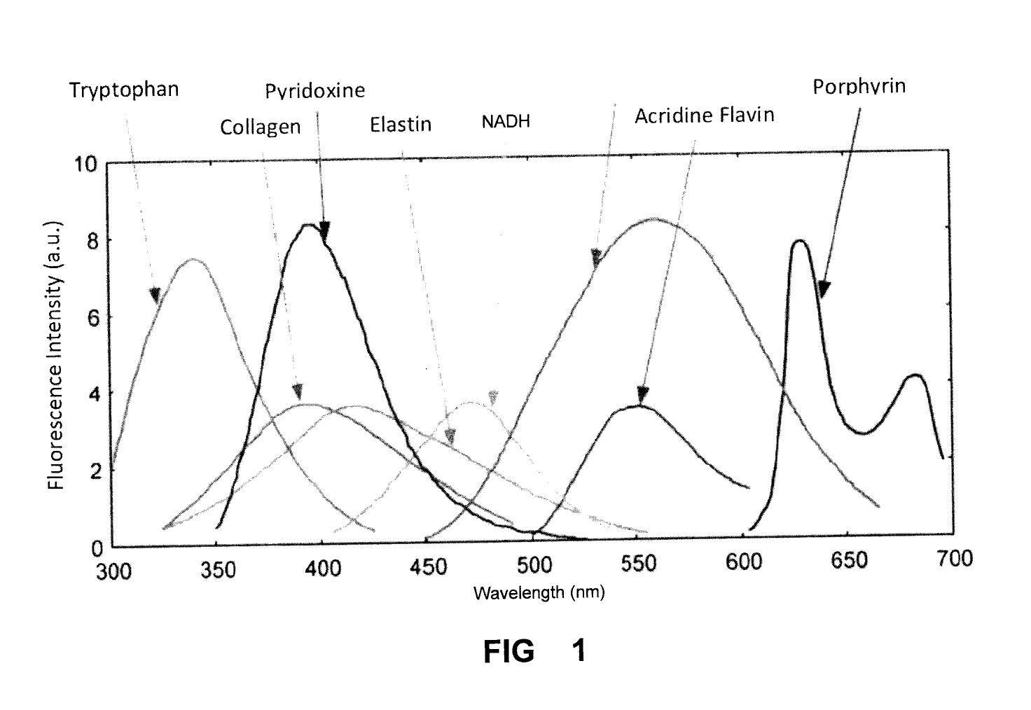

Due to these facts, there arises another tumour detection method different from the morphologic technology. This invention applies the biochemistry based detection which uses the spectrum technology for human early stage tumour detection. The invention is enhanced by the Laser Induced Fluorescence (LIF) technology. Referenced to U.S. Pat. No. 4,957,114 which was transmitted to the applicant of this patent application, the applicant has combined it in the application. The diagnostic method and criteria of LIF have been recognized. Fluorescence spectrum of the biological molecular that are detectable in the epithelia include, bone collagen amino acid, structured protein enzyme and coenzyme, fat and porphyrin metabolism related coenzyme, adenine dinucleotide, flavin adenine dinucleotide (NADH) and flavin nucleic acid, tryptophan, collagen, pyridoxine, elastin, acridine flavin, porphyrin, etc. As FIG. 1 shows, these biological molecular have their own unique spectrum, so the changes of these biological molecular can be expressed in their fluorescence spectrum.

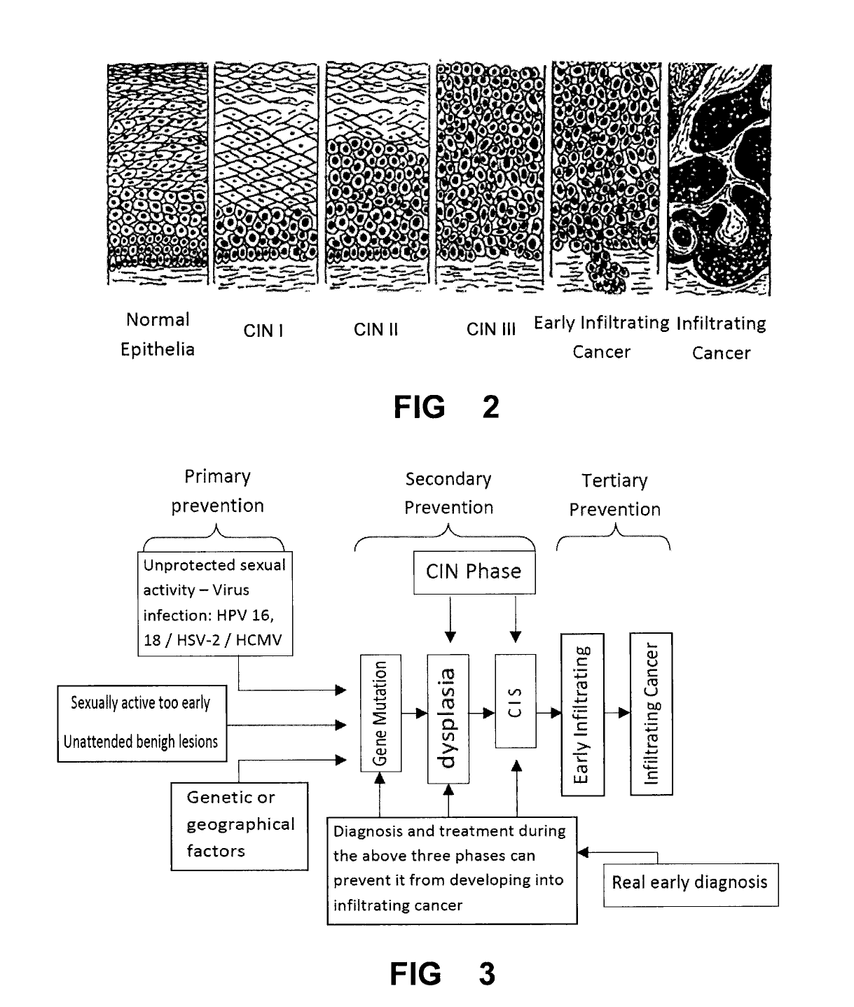

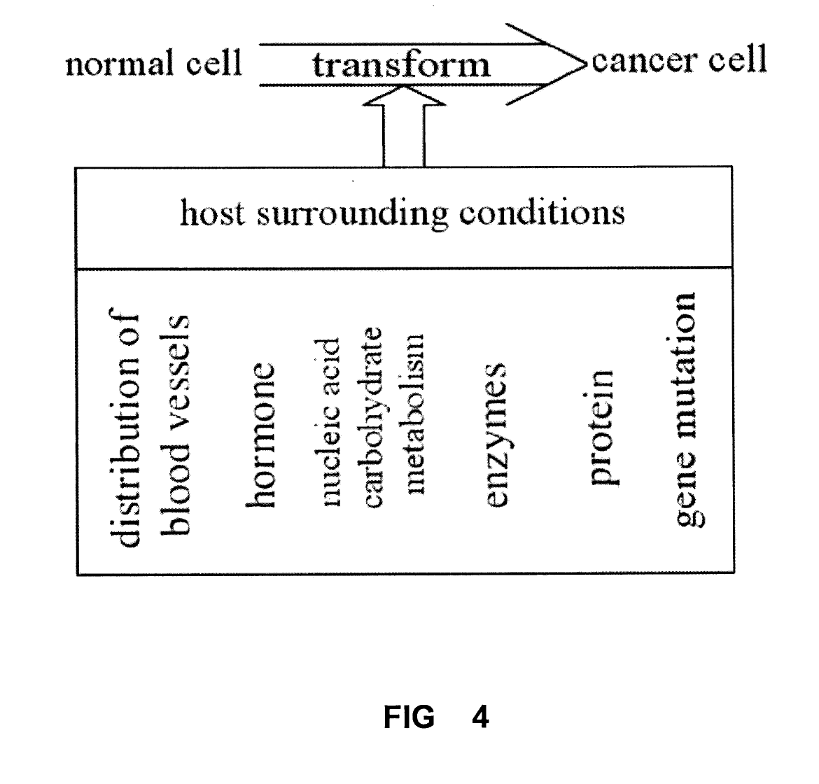

Most malignant tumours are originated in the epithelia with a process of gradual transformation. Cervical cancer is of course one of them. FIG. 2 shows the transformation process of the cells inside epithelia. These include gene mutation phases CIN 1, CIN 2 and CIN 3 (carcinoma in situ), early infiltrating cancer and infiltrating cancer. FIG. 3 shows the formation and development of the cervical cancer. See both FIGS. 2 and 3:

In the initial phase of the cervical cancer there is no qualitative change, except that there are some adverse factors which are:

Unprotected sexual activities induced virus infection. These viruses include HPV 16, HPV 18, HSV-2 and HCMV et al;

Sexually active too early and non attendance to benign lesions;

Genetic or geographical factors.

At this stage there is no qualitative change however the adverse factors may cause disease so the treatment should be mainly preventive or removal the adverse factors. This phase is also a primary preventive phase. It corresponds to ‘normal epithelia’ cells in FIG. 2.

The second phase is the gene mutation. It can be divided into 3 sub-phases, which are: gene mutation (CIN 1), atypical dysplasia (CIN 2) and carcinoma in situ (CIN 3). The transformation of the cells can be referred to CIN 1, CIN 2, and CIN 3 of FIG. 2. The qualitative change has already occurred at the gene mutation phase but no cancer has been formed. Therefore if effective diagnosis and treatment can be given at this phase, it may well be prevented from developing into infiltrating cancer. Ideally early diagnosis should be able to detect the lesions in second phase. However during the gene mutation phase CIN 1, CIN 2 and CIN 3 the transformation all occur within mucous membrane and no obvious morphological characters appear, hence the detection of the change of biochemistry information is very important. The second phase has become a secondary preventive phase.

Referring to FIGS. 2 and 3, in third phase, the cancer has formed, i.e. early infiltrating cancer and infiltrating cancer, which are shown in FIG. 2 as ‘early infiltrating cancer’ and ‘infiltrating cancer’. Because cancer has formed, the treatment now becomes comparatively difficult. The third phase is also called tertiary preventive phase.

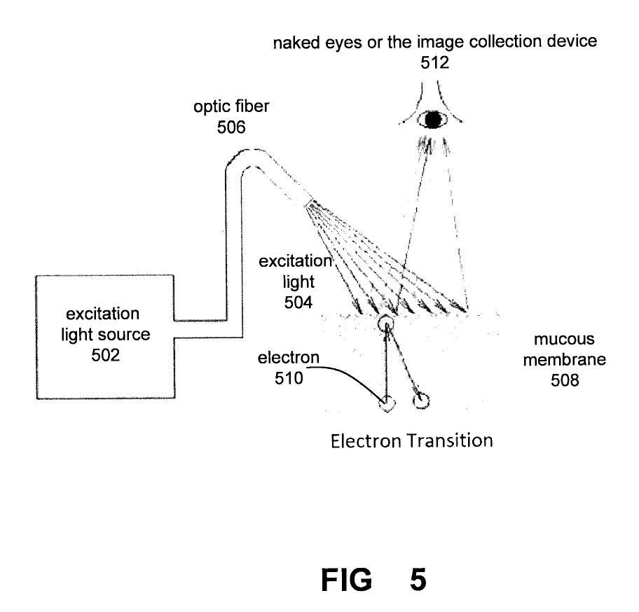

The analysis of FIGS. 2 and 3 has indicated that apparently the key to early diagnosis lies in the second phase. During the cell transformation in the second phase, although the morphological characters of cancer are not quite clear, the change of biochemistry characters is obvious. These biochemistry characters include the early information of biochemistry environment around the host during the transformation of abnormal cells. As shown in FIG. 4, a list of these information are: distribution of blood vessels, hormones, metabolism of nuclear acid and carbohydrates, enzymes, proteins, gene mutation.

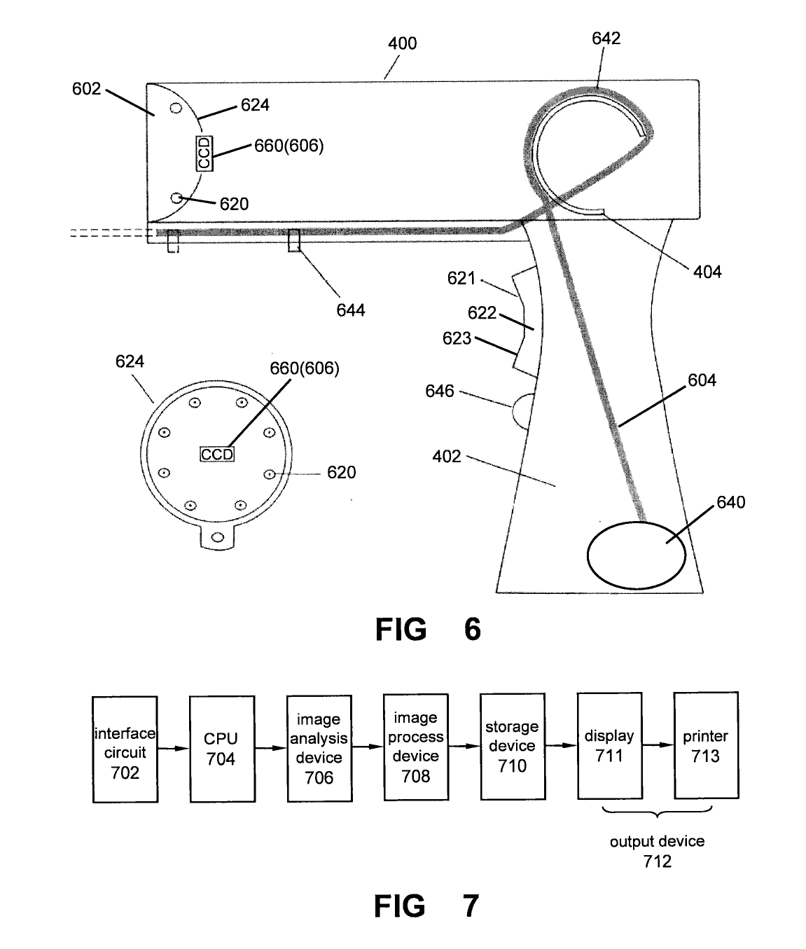

The above mentioned early information all have their own typical spectrum frequency. When the light with a certain frequency irradiates on the epithelia of the cervix mucous membrane, under certain circumstances the electron will absorb the energy and make a transition to a higher energy level (excitation status). If the electron comes back from the excitation status to the basic status by radiation, it will release the energy by giving out the corresponding photon, which results in the generation of the fluorescence (refer to FIG. 5). This is also the basic theory to support LIF cancer diagnosis. As shown in FIG. 5, the excitation light source 502 generates the excitation light 504. Through optic fiber 506 it irradiates on mucous membrane 508. Electron 510 transition within mucous membrane 508 occurs and the respective visible light is presented, which can be observed by naked eyes or the image collection device 512.

Based on the theory above, using light to irradiate the mucous membrane epithelia of the cervix, the fluorescence characters from the epithelia can be used to judge if it's normal tissue, benign lesion, CIN or cancer. They can be presented with fluorescent spectrum or fluorescent images. This invention will make the judgements by the fluorescent images. Doctors can screen the patients by differentiating the colours of fluorescence from observation of naked eyes or from images on a monitor. The advantages of using this method are: 1) high detection rate of CIN—CIN may be detected even at deep locations of the mucous membrane epithelia of the cervix; 2) low cost—only 2 Chinese Yuan for each patient screening; 3) fast screening—only 5 minutes for each patient. In addition, if a CCD image recording device is used, the images taken can be processed by the computer to perform automatic image recognition, storage, et al. This method is suitable to be used for diagnosis in hospitals.

TABLE 1
shows the criteria of CIN phase differentiation

| Type | Colour of measured mucous membrane |
|---|---|
| Normal tissue | Bluish white |
| Benign lesions | Orangish yellow or orangish red |
| CIN 3 | Purplish red |
| CIN 2 | Dark purple or dark red |
| CIN 1 | Dark |

On the basis of the fluorescence image diagnosis, this invention also introduced acetic acid coloring image diagnosis. Therefore, the core of this invention is that it united the inherent fluorescence image diagnosis and acetic acid coloring image diagnosis. If used individually, either can reach 40% positive CIN diagnosis. The reason is that fluorescence image diagnosis is made from the biochemical changes within epithelia of cervical mucous membrane, while the acetic acid coloring image method (visual inspection with acetic acid, VIA) demonstrates the change of the outer layer of epithelia of cervical mucous membrane (keratinized layer). So the locations suitable for these two are slightly different. As the CIN occur nearly half within the epithelia of mucous membrane and the other half at outer layer of epithelia of mucous membrane, if only using one method alone, some lesions won't be detected. However if these two methods are united, generally they will have a full coverage of all lesions of the epithelia of cervical mucous membrane. That is, VIA covering the detection of outer layer of epithelia and fluorescence image covering the detection within epithelia. The combination of these two diagnostic methods can improve the detection rate of cervical precancerous lesions to 70%-80%. Normal cervical squamous epithelium with less protein is suitable for fluorescence detection of biochemical information within the epithelia. However metaplasia epithelium, and the membrane, nucleus and cytoplasm of the poorly differentiated epithelial cell contain more collagen and elastin, so the fluorescent characters are covered by the superficial protein and important intraepithelial biochemical information can't be revealed by the fluorescence diagnosis. NADH (Nicotinamicle adeninedinucleotide) is an example. NADH is considered antioxidants which has a protective effect to free radical oxidation. The decreasing of NADH can cause cell damage and will be easier for cancer to form. NADH fluorescence is peaked at around 470 nm, so the 470 nm peak values can be used as evidence to justify if it's cancerous tissue or not. If the outer layer of cervical epithelia (keratinized layer) is covered by collagen and elastin, then the fluorescence data acquired must be fluorescence from these proteins, while the fluorescence of important biochemical substances within epithelia such as NADH can not be displayed. This is the blind spot of fluorescence diagnosis. The VIA is able to scientifically fill this blind spot. Combined use of these two methods significantly increased positive detection rate of CIN, which was confirmed by the data of a large population sample screening for CIN.

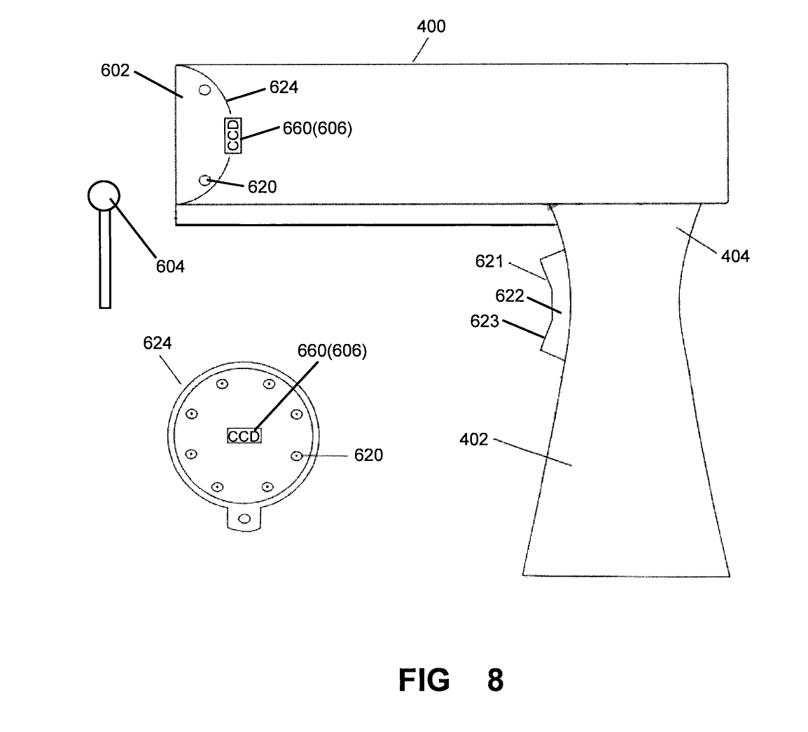

FIG. 6 shows the first type of implementation structure of the portable diagnostic device for cervical precancerous lesion in this invention. This portable device 600 includes:

The inherent fluorescence detection system for cervical cancer 602, including light source 620 and switch 622, which consists of the excitation light source that generates the fluorescence and the cold light source that generates the white light. Both of light sources are installed in the bowl-shaped reflector 624 with specific focal length. Light from light sources is focused to cervix by the bowl-shaped reflector 624. The wave length of the excitation light is 360-440 nm. Accordingly, the following light sources can be used as the excitation light: laser instrument that can produce a spectrum range of 360-440 nm, LED that can produce a spectrum range of 360-440 nm, high-pressure mercury light that can produce a spectrum range of 360-440 nm, or Xenon light that can produce a spectrum range of 360-440 nm. If using laser instrument or high-pressure mercury light as the excitation light source, because their size is comparatively large, they are placed outside the portable cervical precancerous lesion diagnostic device 600. Their light is introduced through quartz optic fiber, liquid optic fiber or ordinary optic fiber to the bowl-shaped reflector 624. The surface of the bowl-shaped reflector 624 is painted with hard or soft membrane that can reflect the ultraviolet light. The focal length of the bowl-shaped reflector 624 is about 120 mm.

This portable cervical precancerous lesion diagnostic device 600 also includes: Cervix acetic acid coloring detection system 604 with acetic acid pool 640, spraying tube 642 that connects with the acetic acid pool, spraying tube moving device 644 that is used to move spraying tube 642, and spraying switch 646 that controls the on/off of the spraying tube 642.

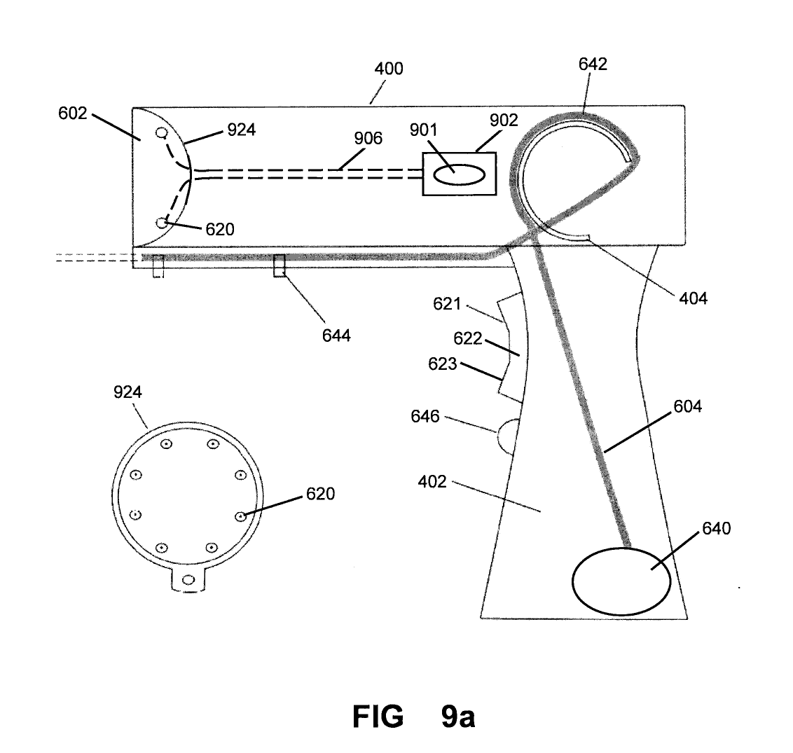

As shown in FIG. 7, this portable cervical precancerous lesion diagnostic device 600 is fixed on a pistol shape stand. The pistol shape stand consists of a horizontal part 400 and a handle 402. The back end of the horizontal part 400 connects with the upper end of the handle 402. Bowl-shaped reflector 624 is at the front of the horizontal part 400. The light sources 620 are installed on the bowl-shaped reflector 624 with same distance apart to form a ring. The acetic acid pool 640 is inside the handle 402. The spraying tube 642 extends from acetic acid pool 640 and goes up inside the handle 402. After passing a direction switch 404 the spraying tube continues along beneath the horizontal part 400 to extend to the front end inside the horizontal part. The spraying tube direction switch 404 has the structure like a ring. The spraying tube 642 makes one circle around the direction switch 404 then its extension direction changes from vertical to horizontal. The spraying tube moving device 644 can be installed on the horizontal part 400 with some flexibility. The spraying tube moving device 644 reaches beyond the horizontal part 400 and connects with the spraying tube 642 inside the horizontal part 400. The spraying tube moving device 644 can drive the front end of the protruded horizontal part 400. Generally, spraying tube 642 can reach out for about 5 cm from the tip of the horizontal part 400. The light source switch 622 is installed on the handle 402. This 622 includes the excitation light source switch 621 and cold light source switch 623. As shown in FIG. 6, in an embodiment, the light source switch 622 is made as a two way switch. The upper part is the excitation light source switch 621, while the lower part is the cold light source switch 623. The spraying switch 646 is also installed on the handle 402.

As shown in FIG. 6, in an embodiment, there is also the image collection system 606. This image collection system includes weak light CCD 660, which is installed in the center of the bowl-shaped reflector 624 to collect the light reflected from the cervix. The image collection system 606 is connected to the external image processing system (as shown in FIG. 7) which then process the image collected.

As shown in FIG. 7, in an embodiment adopts an external image processing system 700 which includes: Interface circuit 702, weak light CCD 660 that connects to the image collection system 606. CPU 704 is connected to the interface circuit 702 and receives the images collected by the weak light CCD 660. The image analysis device 706, which is connected to the CPU 704, will analyze the image that is transmitted by CPU 704. Image process device 708, which is connected to the image analysis device 706, will process the analyzed images. Storage device 710, which is connected to the image processing device 708, will store the images and the processed results. Output device 712 will output the images and processed results. Output device can include display 711 and printer 713.

As shown in FIG. 6, the working procedures of the embodiment are as the following: First is the fluorescence detection. In the above mentioned inherent fluorescence detection system 602, use the white light generated by cold light source and excitation light generated by the excitation light source to irradiate the cervix in turn. The image collection system 606 collects reflected images from the cervix induced by white and excitation light then transmits these images to the external image processing system 700. Then it's the acetic acid detection. Cervical acetic acid coloring detection system 604 sprays acetic acid on cervix; Wait for a period of time and use the white light and excitation light from inherent fluorescence detection system 602 to irradiate the cervix in turn; The image collection system 606 collects reflected images from the acetic acid sprayed cervix induced by white light and excitation light irradiation and then transmits these images to the external image processing system 700.

In an embodiment, the operation of portable diagnostic device for cervical precancerous lesions is as follows: after the speculum is placed in patient, the doctor will put the front end of the horizontal part of the portable diagnostic device for cervical precancerous lesions approximately 30 mm to the opening of the speculum, switch on the cold light source, identify the cervical status under white light through the CCD, then turn off all lights in the diagnostic room (like a chamber) and switch on the excitation light source, observe cervical inherent fluorescence to identify lesions through CCD, and with reference to Table 1 Diagnostic Criteria to determine cervical status of the patients. Later turn off the excitation light and turn on the white light, move the spraying tube moving device to relocate it to a certain distance (such as 5 cm) and aim to cervix of the patient, press the spray switch to spray 0.5 ml acetic acid to cervix of the patient, after 2-3 minutes under white light identify through the CCD if patient has acetic acid colored white epithelia. Through the CCD, cervix status can be seen directly on the display, while CCD will also transmit the received signals through the interface circuit to the CPU, after image analysis and image processing a diagnostic report will be made and seen on the display or stored in a storage device or printed from a printer.

If using weak Light CCD with portable diagnostic devices for cervical precancerous lesions, the accuracy of detection rate is higher, but relatively speaking, CCD and its auxiliary equipment required a larger space and are more complex. Therefore it's more suitable as a standard exam in the hospital.

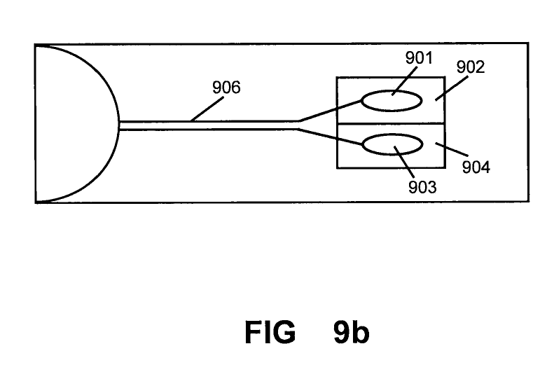

FIG. 8 reveals a second embodiment of the portable diagnostic device for cervical precancerous lesions in this invention. Compared to the first implementation as shown in FIG. 6, the difference in the second implementation is that the cervical acetic acid coloring detection system was abolished. It uses separated cotton sticks or cotton balls 604 to smear the acetic acid. In practice, firstly using white light to irradiate, followed by use of cotton sticks or cotton balls to smear acetic acid for coloring, and then to irradiate with excitation light. The embodiment as shown in FIG. 8 still uses weak light CCD, which should also be more suitable for standard exam in the hospital.

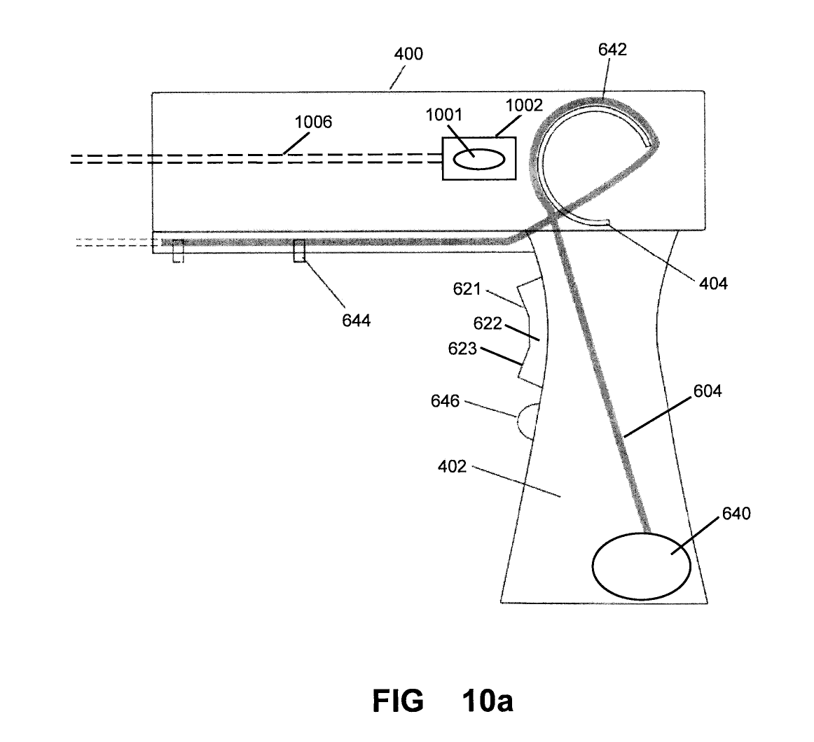

FIGS. 9a and 9b reveal a third embodiment of the portable diagnostic device for cervical precancerous lesions in this invention. In this embodiment, the image collection system was abolished. The weak light CCD is no longer placed at the centre of the bowl-shape reflector 924. The reflected light is observed with the naked eyes of a doctor. The rest of the structure are the same as shown in FIG. 6. Although not using CCD would compromise the detection accuracy slightly, the speed of detection can be greatly increased. In addition, without CCD and image processing equipment, the size of the diagnostic device is greatly reduced and its mobility is improved, which should be suitable for large population screening in the movement.

In an embodiment as shown in FIGS. 9a and 9b, the excitation light source is excitation light LED 901. The cold light source is white light LED 903. Excitation light LED and white light LED are installed in the separated darkrooms 902 and 904 (refer to 9b) of the portable diagnostic device for cervix precancerous lesions, respectively. The input end of a fork-like quartz or liquid optic fiber 906 is connected to the excitation light LED 901 and the white light LED 903, respectively. The output end of the fork-like quartz or liquid optic fiber 906 is covered by the metal or the plastic overcoat. In the embodiment of 9a and 9b, CCD is not used. It will also be feasible if the above light source is incorporated to the first or second embodiment.

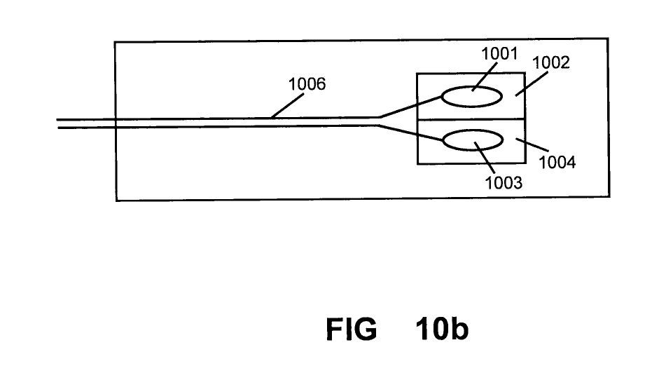

FIGS. 10a and 10b reveal a fourth embodiment of the portable diagnostic device for cervical precancerous lesions in this invention. The fourth embodiment is the further change of the third embodiment as shown in FIGS. 9a and 9b. The bowl-shaped reflector is also abolished. In FIGS. 10a and 10b, the excitation light source is excitation light LED 1001. The cold light source is white light LED 1003. Excitation light LED and white light LED are installed in the separated darkrooms 1002 and 1004 (refer to 10b) of the portable diagnostic device for cervix precancerous lesions, respectively. The input end of a fork-like quartz or liquid optic fiber 1006 is connected to the excitation light LED 1001 and the white light LED 1003, respectively. The output end of the fork-like quartz or liquid optic fiber 1006 is covered by the metal or the plastic overcoat. The output end of the fork-like quartz or liquid optic fiber 1006 extends beyond the front end of the portable diagnostic device for cervix precancerous lesions. Because the diameter of the fork-like quartz or liquid optic fiber 1006 is smaller, it's easier for the doctor to observe by naked eyes. The embodiment shown in FIGS. 10a and 10b is highly mobile, which is also suitable for quick screening of large population in the movement.

Although the invention were described with the above embodiments, it should be noted that in practice, the inherent fluorescence image detection device and acetic acid coloring detection are not necessarily placed within one operation stand. It is completely acceptable to place them separately. In addition, other devices that have combined application of inherent fluorescence image detection or cervical acetic acid coloring detection should be considered within the scope of the invention, although they may not be the same form that is displayed in this invention. For technical personnel in this field, obviously, the invention has presented a mucosal cancer detection method with high accuracy through a combination of inherent fluorescence imaging detection method and the cervical acetic acid coloring detection method.

As described above, since the developments of most mucous type precancerous lesions prior to mucosal cancers are similar, the device of the invention can also be used for the diagnosis of other mucous type precancerous lesions.

The invention combines the inherent fluorescence image detection method and cervical acetic acid coloring detection method. It has greatly increased the cervical precancerous lesions (CIN) detection rate, which can reach approximately 80%. The diagnostic method of the invention is generally based on objective evidence. It's less dependent on experience. Even a junior doctor can detect CIN correctly. Also, the test used for detection is low-cost and less time consuming. Detection of a patient costs only 2 Yuan and the detection time is only about 5 minutes. The invention of the portable diagnostic device for cervical precancerous lesions can help to identify more cervical precancerous lesions. After approved treatment the lesion is able to be blocked in the direction to develop to cervical cancer. This will also directly reduce the incidence of cervical cancer.

Although the ideal embodiments are described as above, the invention is not limited to these examples. The skilled technicians of this field can make changes and amendments on the basis of the above descriptions. Various changes and amendments not deviating from the invention should all fall within the scope of protection. Scope of protection of the invention should be limited by the appended rights claim.

What is claimed is:

1. A portable diagnostic device for cervical precancerous lesions, comprising:
an inherent fluorescence detection system for cervical cancer, with a light source and a switch, consisting of a excitation light source that generates fluorescence and a cold light source that generates white light, both the excitation light source and the cold light source being installed in a bowl-shaped reflector with specific focal length;
a cervix acetic acid coloring detection system with an acetic acid pool, a spraying tube that connects with the acetic acid pool, a spraying tube moving device for moving the spraying tube, and a spraying switch for controlling the spraying tube;
wherein the inherent fluorescence detection system for cervical precancerous lesions uses the white light and excitation light to irradiate the cervix in turn, and the cervix acetic acid coloring system sprays acetic acid on cervix and uses the white light and excitation light to irradiate the cervix in turn after waiting for a period of time and.

2. A portable diagnostic device for cervical precancerous lesions, comprising:
an inherent fluorescence detection system for cervical cancer, with a light source and a switch, consisting of a excitation light source that generates fluorescence and a cold light source that generates white light, both the excitation light source and the cold light source being installed in a bowl-shaped reflector with specific focal length, light from the light sources being focused to cervix by the bowl-shaped reflector;
a cervix acetic acid coloring detection system with an acetic acid pool, a spraying tube that connects with the acetic acid pool, a spraying tube moving device for moving the spraying tube, and a spraying switch for controlling the spraying tube;
an image collection system with a weak light CCD, installed in the center of the bowl-shaped reflector to collect the light reflected from the cervix, the image collection system being connected to the external image processing system which then process the image collected;
wherein the inherent fluorescence detection system for cervical precancerous lesions uses the white light and excitation light to irradiate the cervix in turn, the image collection system collects reflected images from the cervix induced by white and excitation light then transmits these images to the external image processing system, the cervix acetic acid coloring system sprays acetic acid on cervix and uses the white light and excitation light to irradiate the cervix in turn after waiting for a period of time, the image collection system collects reflected images from the cervix induced by acetic acid spray then transmits these images to the external image processing system.

3. The portable diagnostic device for cervical precancerous lesions according to claim 2, wherein the device is fixed on a pistol shape stand, the pistol shape stand consists of a horizontal part and a handle, the back end of the horizontal part connects with the upper end of the handle;
the bowl-shaped reflector is at the front of the horizontal part, the light sources are installed on the bowl-shaped reflector with same distance apart to form a ring;
the acetic acid pool is inside the handle, the spraying tube extends from acetic acid pool to the top of the handle, after passing a direction switch the spraying tube continues along beneath the horizontal part to extend to the front end inside the horizontal part;
the spraying tube moving device is flexibility installed on the horizontal part, the spraying tube moving device reaches beyond the horizontal part and connects with the spraying tube inside the horizontal part, the spraying tube moving device drives the front end of the protruded horizontal part;
the light source switch is installed on the handle, which includes the switches of excitation light source and cold light source;
the spray switch is installed in the handle.

4. The portable diagnostic device for cervical precancerous lesions according to claim 2, wherein the wavelength of excitation light source is 360-440 nm, the excitation light sources include:
a laser instrument that produces a spectrum range of 360-440 nm, LED that produces a spectrum range of 360-440 nm, high-pressure mercury light that produces a spectrum range of 360-440 nm, or Xenon light that produces a spectrum range of 360-440 nm.

5. The portable diagnostic device for cervical precancerous lesions according to claim 4, wherein the excitation light source directs the light into the bowl-shaped reflector through a quartz optic fiber, a liquid optic fiber or an ordinary optic fiber.

6. The portable diagnostic device for cervical precancerous lesions according to claim 2, wherein the surface of the bowl-shaped reflector is covered by the hard or soft membrane that reflects ultraviolet light.

7. The portable diagnostic device for cervical precancerous lesions according to claim 2, wherein the external image processing system includes:
an interface circuit, connecting with the weak light CCD in the image collection system;
a CPU connected to the interface circuit for receiving the images collected by the weak light CCD;
an image analysis device connected to the CPU for analyzing the image transmitted by CPU;
an image process device connected to the image analysis device for processing the analyzed images;
a storage device connected to the image processing device for storing the images and the processed results;
an output device for outputting the images and processed results.

8. A portable diagnostic device for cervical precancerous lesions, comprising:
an inherent fluorescence detection system for cervical cancer, with a light source and a switch, consisting of a excitation light source that generates fluorescence and a cold light source that generates white light, both the excitation light source and the cold light source being installed in a bowl-shaped reflector with specific focal length, light from the light sources being focused to cervix by the bowl-shaped reflector;
cotton sticks or cotton balls for smearing the acetic acid to the cervix;
an image collection system with a weak light CCD, installed in the center of the bowl-shaped reflector to collect the light reflected from the cervix, the image collection system being connected to the external image processing system which then process the image collected;
wherein the inherent fluorescence detection system for cervical precancerous lesions uses the white light and excitation light to irradiate the cervix in turn, the image collection system collects reflected images from the cervix induced by white and excitation light then transmits these images to the external image processing system, use the cotton sticks or cotton balls to smear acetic acid on cervix and use the white light and excitation light to irradiate the cervix in turn after waiting for a period of time; the image collection system collects reflected images from the cervix induced by smeared acetic acid and then transmits these images to the external image processing system.

9. The portable diagnostic device for cervical precancerous lesions according to claim 8, wherein:
the excitation light source is excitation light LED; the cold light source is white light LED; the excitation light LED and the white light LED are installed in separated darkrooms of the portable diagnostic device, respectively; the input end of a fork-like quartz or liquid optic fiber is connected to the excitation light LED and the white light LED, respectively; the output end of the fork-like quartz or liquid optic fiber is covered by the metal or the plastic overcoat.

10. A portable diagnostic device for cervical precancerous lesions, comprising:
an inherent fluorescence detection system for cervical cancer, with a light source and a switch, consisting of a excitation light source that generates fluorescence and a cold light source that generates white light, the excitation light source and the cold light source being connected to the input end of a quartz or liquid optic fiber which is covered by the metal or plastic overcoat, the output end of the quartz and liquid optic fiber irradiate straight to the cervix;
a cervix acetic acid coloring detection system with an acetic acid pool, a spraying tube that connects with the acetic acid pool, a spraying tube moving device for moving the spray tube, and a spraying switch for controlling the spraying tube;
wherein the inherent fluorescence detection system for cervical precancerous lesions uses the white light and excitation light to irradiate the cervix in turn, the cervix acetic acid coloring system sprays acetic acid on cervix and uses the white light and excitation light to irradiate the cervix in turn after waiting for a period of time.

11. The portable diagnostic device for cervical precancerous lesions according to claim 1, wherein:
the excitation light source is excitation light LED; the cold light source is white light LED; the excitation light LED and the white light LED are installed in separated darkrooms of the portable diagnostic device, respectively; the input end of a fork-like quartz or liquid optic fiber is connected to the excitation light LED and the white light LED, respectively; the output end of the fork-like quartz or liquid optic fiber is covered by the metal or the plastic overcoat.

12. The portable diagnostic device for cervical precancerous lesions according to claim 2, wherein:
the excitation light source is excitation light LED; the cold light source is white light LED; the excitation light LED and the white light LED are installed in separated darkrooms of the portable diagnostic device, respectively; the input end of a fork-like quartz or liquid optic fiber is connected to the excitation light LED and the white light LED, respectively; the output end of the fork-like quartz or liquid optic fiber is covered by the metal or the plastic overcoat.
